(19) United States
(12) Patent Application Publication
(10) Pub. No.: US 2022/0005338 A1
(43) Pub. Date: Jan. 6, 2022

(54) PERSONAL SAFETY ASSURANCE SYSTEM AND SAFETY MONITORING METHOD

(71) Applicant: ZHEJIANG NORMAL UNIVERSITY, Zhejiang (CN)

(72) Inventors: Lina CHEN, Zhejiang (CN); Yumeng TAN, Zhejiang (CN); Mao LIU, Zhejiang (CN); Xupeng FU, Zhejiang (CN); Lianchao HE, Zhejiang (CN); Jianbo ZHU, Zhejiang (CN)

(21) Appl. No.: 17/276,326

(22) PCT Filed: Jul. 29, 2020
(86) PCT No.: PCT/CN2020/105443

§ 371 Date: Mar. 15, 2021

(30) Foreign Application Priority Data
Dec. 26, 2019 (CN) 201911365307.0

(51) Int. Cl. G08B 21/04; G08B 25/10; H04W 4/90; A61B 5/021; A61B 5/00

(52) U.S. Cl. CPC G08B 21/0453; G08B 25/10; A61B 5/6831; A61B 5/021; H04W 4/90

(57) ABSTRACT

The present disclosure relates to a personal safety assurance system and a safety monitoring method. A smart band includes a detection module, a main chip, a band emergency button, and a wireless band connection module. The smart band transmits detected heart rate, blood pressure, and pulse of a human body to a safety monitoring application (APP). The band emergency button is configured to receive first emergency alarm information, and transmit the first emergency alarm information to the main chip. The main chip is further configured to transmit the first emergency alarm information to the safety monitoring APP through the wireless band connection module. The safety monitoring APP is configured to display the heart rate, the blood pressure, and the pulse, transmit a physical abnormality alarm signal to a hospital alarm platform, and transmit the first emergency alarm information to a public security alarm platform.

The present application claims priority to the Chinese Patent Application No. 201911365307.0, filed with the China National Intellectual Property Administration (CNIPA) on Dec. 26, 2019, and the International Patent Application No. PCT/CN2020/105443 filed Jul. 29, 2020 and entitled “PERSONAL SAFETY ASSURANCE SYSTEM AND SAFETY MONITORING METHOD AND SYSTEM”, which is incorporated herein by reference in its entirety for whatever purpose.

### TECHNICAL FIELD

The present disclosure relates to the technical field of automatic control, and specifically, to a personal safety assurance system and a safety monitoring method.

### BACKGROUND

As the society and economy develop, and work and life styles change, people have a fast pace of life and spend more time alone. However, staying alone blocks messages. For example, a young writer was dead due to a sudden illness for more than ten days before the writer's body was discovered; a young girl who hailing a taxi alone could not ask for help in time, resulting in a tragedy. Similar news frequently entered the public view. At present, in view of personal safety issues, there is no monitoring system that can comprehensively guard personal safety.

### SUMMARY

The present disclosure aims to provide a personal safety assurance system and a safety monitoring method, so as to comprehensively guard personal safety.

To achieve the objective, the present disclosure provides the following solutions:

A personal safety assurance system is provided. The assurance system includes a smart band and a safety monitoring application (APP).

The smart band includes a detection module, a main chip, a band emergency button, and a wireless band connection module.

An output end of the detection module is connected to an input end of the main chip. The detection module is configured to detect a heart rate, blood pressure, and a pulse of a human body, and transmit the heart rate, the blood pressure, and the pulse to the main chip. An output end of the main chip is connected to the safety monitoring APP through the wireless band connection module. The main chip is configured to transmit the heart rate, the blood pressure, and the pulse to the safety monitoring APP through the wireless band connection module. The safety monitoring APP is configured to display the heart rate, the blood pressure, and the pulse, and convert the heart rate, the blood pressure, and the pulse into a physical abnormality alarm signal.

A signal output end of the band emergency button is connected to the input end of the main chip. The band emergency button is configured to send an emergency alarm signal to the main chip. The main chip is further configured to transmit the emergency alarm signal to the safety monitoring APP through the wireless band connection module.

The safety monitoring APP is connected to a public security alarm platform and a hospital alarm platform through a network. The safety monitoring APP is further configured to transmit the physical abnormality alarm signal to the hospital alarm platform and an emergency contact's mobile phone, generate emergency alarm information based on the emergency alarm signal, and transmit the emergency alarm information to the public security alarm platform and the emergency contact's mobile phone.

A safety monitoring method is provided. The monitoring method includes:

obtaining a heart rate, blood pressure, and a pulse;

comparing the heart rate, the blood pressure, and the pulse with a preset standard to obtain a comparison result;

determining whether the comparison result is in a comparison deviation range to obtain a first determining result;

if the first determining result indicates that the comparison result goes beyond the comparison deviation range, generating a physical abnormality alarm signal, and sending the physical abnormality alarm signal to an emergency contact's mobile phone and a hospital alarm platform;

determining whether emergency alarm information is received to obtain a second determining result, where the emergency alarm information includes first emergency alarm information, second emergency alarm information, or third emergency alarm information; and

if the second determining result indicates that the emergency alarm information is received, sending the emergency alarm information to the emergency contact's mobile phone and a public security alarm platform.

Optionally, the determining whether emergency alarm information is received to obtain a second determining result specifically includes the following:

if an alarm signal sent by a smart band is received, the second determining result is that the first emergency alarm information is received;

if a voice message of a user is received, the voice message is matched with a preset emergency voice key, and if the matching succeeds, the second determining result is that the second emergency alarm information is received; or

if a simulated call instruction of the user is received, dialog content is matched with the preset emergency voice key, and if the matching succeeds, the second determining result is that the third emergency alarm information is received.

Optionally, after the sending the physical abnormality alarm signal to an emergency contact's mobile phone and a hospital alarm platform, the method further includes:

obtaining first positioning information, and sending the first positioning information to the emergency contact's mobile phone and the hospital alarm platform.

Optionally, after the sending the emergency alarm information to the emergency contact's mobile phone and a public security alarm platform, the method further includes:

obtaining second positioning information, and sending the second positioning information to the emergency contact's mobile phone and the public security alarm platform; and

obtaining video information and audio information, and sending the video information and the audio information to the emergency contact's mobile phone and the public security alarm platform.

The present disclosure has the following beneficial effects:

The present disclosure discloses the personal safety assurance system and the safety monitoring method. The safety monitoring APP determines whether the user is physically abnormal based on the user's heart rate, blood pressure, and pulse detected by the smart band in real time, and sends the alarm information to the hospital alarm platform and the emergency contact's mobile phone when the user is physically abnormal. In addition, when an emergency occurs, the user can press the emergency button on the smart band to send the alarm information to the public security alarm platform and the emergency contact's mobile phone. This comprehensively guards personal safety.

### BRIEF DESCRIPTION OF DRAWINGS

To more clearly describe the technical solutions in the embodiments of the present disclosure or in the prior art, the following describes the accompanying drawings to be used in the embodiments. Apparently, the accompanying drawings in the following description show merely some embodiments of the present disclosure, and a person of ordinary skill in the art may further derive other drawings from these accompanying drawings without creative efforts.

FIG. 1 is a schematic structural diagram of a personal safety assurance system according to the present disclosure;

FIG. 2 is a flowchart of a safety monitoring method according to the present disclosure;

FIG. 3 is a schematic diagram of a personal safety assurance system according to the present disclosure;

FIG. 4 is a schematic diagram of a daily mode of a personal safety assurance system according to the present disclosure; and

FIG. 5 is a schematic diagram of an abnormality determining mode of a personal safety assurance system according to the present disclosure.

Reference numeral description: 1. smart band and 2. safety monitoring APP.

### DETAILED DESCRIPTION

The technical solutions in the embodiments of the present disclosure are clearly and completely described below with reference to the accompanying drawings. Apparently, the described embodiments are merely some rather than all of embodiments of the present disclosure. All other embodiments obtained by a person of ordinary skill in the art based on the embodiments of the present disclosure without creative efforts shall fall within the protection scope of the present disclosure.

An objective of the present disclosure is to provide a personal safety assurance system and a safety monitoring method, so as to comprehensively guard personal safety.

To make the above objective, features, and advantages of the present disclosure clearer and more comprehensible, the present disclosure is further described in detail below with reference to the accompanying drawings and specific embodiments.

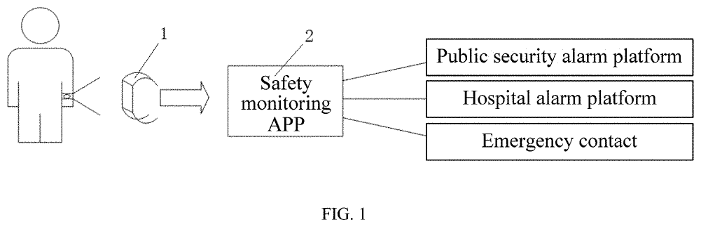

FIG. 1 is a schematic structural diagram of a personal safety assurance system according to the present disclosure. As shown in FIG. 1, the assurance system includes a smart band 1 and a safety monitoring application (APP) 2.

The smart band 1 includes a detection module, a main chip, a band emergency button, and a wireless band connection module.

An output end of the detection module is connected to an input end of the main chip. The detection module is configured to detect a heart rate, blood pressure, and a pulse of a human body, and transmit the heart rate, the blood pressure, and the pulse to the main chip. An output end of the main chip is connected to the safety monitoring APP 2 through the wireless band connection module. The main chip is configured to transmit the heart rate, the blood pressure, and the pulse to the safety monitoring APP 2 through the wireless band connection module. The safety monitoring APP 2 is configured to display the heart rate, the blood pressure, and the pulse, and convert the heart rate, the blood pressure, and the pulse into a physical abnormality alarm signal. A user's health status data is updated in real time on the safety monitoring APP 2. The heart rate, the blood pressure, and the pulse of the human body that are detected by the smart band 1 are further transmitted to a cloud to establish a personal health database. Preferably, the smart band 1 is connected to the safety monitoring APP 2 through Bluetooth.

A signal output end of the band emergency button is connected to the input end of the main chip. The band emergency button is configured to receive an emergency alarm signal, and transmit the emergency alarm signal to the main chip. The main chip is further configured to transmit the emergency alarm signal to the safety monitoring APP 2 through the wireless band connection module.

The safety monitoring APP 2 is connected to a public security alarm platform and a hospital alarm platform through a network. The safety monitoring APP 2 is further configured to transmit the physical abnormality alarm signal to the hospital alarm platform and an emergency contact's mobile phone, generate emergency alarm information based on the emergency alarm signal, and transmit the emergency alarm information to the public security alarm platform and the emergency contact's mobile phone.

The personal safety assurance system provided in the present disclosure may operate in a daily mode or an abnormality determining mode.

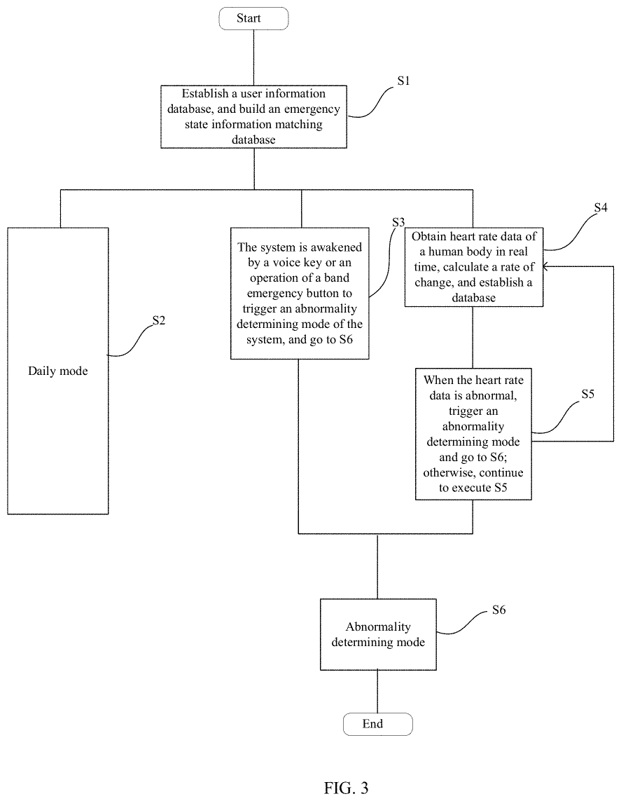

As shown in FIG. 3, after the personal safety assurance system starts to run, S1 is first executed, that is, a user information database is established, and an emergency state information matching database is built. Then S2 is executed, that is, the system operates in the daily mode. When the user says an emergency voice key or presses the band emergency button, S3 is executed, that is, the system is awakened, and an abnormality determining mode of the system is triggered. Then S6 is executed. After S1, the smart band collects the user's heart rate data in real time, calculates a rate of change, and establishes a database, that is, S4. When the system detects abnormal heart rate data, S5 is executed to trigger the abnormality determining mode of the system. Then S6 is performed.

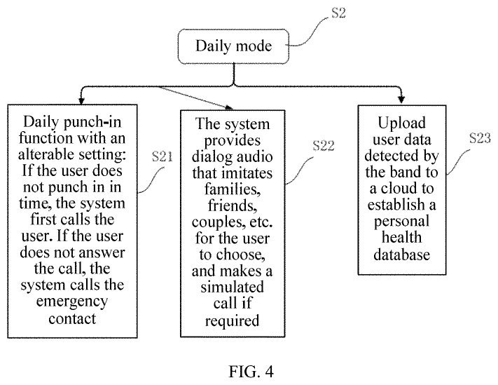

The daily mode in S2 includes a daily punch-in function S21, call simulation S22, and database establishment S23, as shown in FIG. 4. A setting of the daily punch-in function can be altered. Daily punch-in time is set on the safety monitoring APP. The system is automatically awakened ten minutes before the punch-in time, and sends a message to remind the user to punch in. The band vibrates if the punch-in time approaches. If the user does not punch in in time, the system first calls the user. If the user does not answer the call, the system calls the emergency contact and asks the emergency contact to confirm the user's safety. The safety monitoring APP can provide dialog audio that imitates relatives, friends, couples, etc. for the user to choose. The smart band detects the user's heart rate, blood pressure, and pulse in real time, and transmits the detected heart rate, blood pressure, and pulse data to the cloud to establish the personal health database.

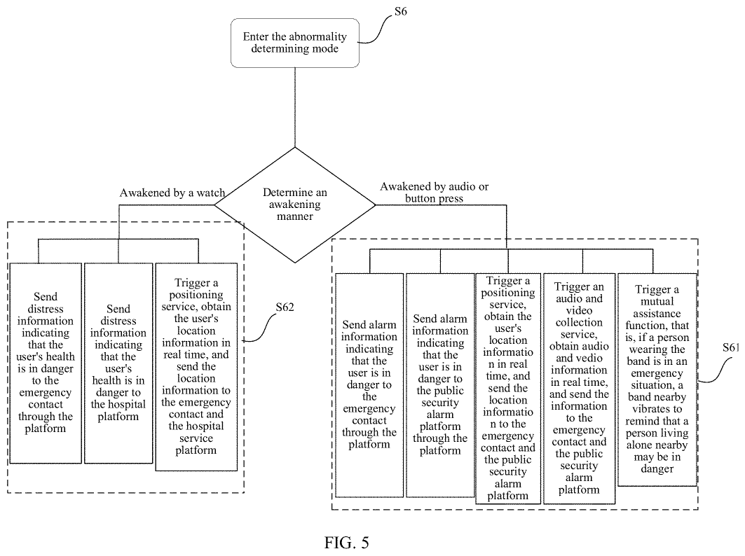

As shown in FIG. 5, after S6 is executed, that is, the system enters the abnormality determining mode, a manner of awaking the system is determined. If the system is awaken by a watch (S5), S62 is executed, including the following: the safety monitoring APP sends distress information indicating that the user's health is in danger to the emergency contact; the safety monitoring APP sends the distress information indicating that the user's health is in danger to the hospital alarm platform; and trigger a positioning service, that is, the safety monitoring APP obtains the user's location information in real time and sends the location information to the emergency contact and the hospital alarm platform. When the system is awaken by audio (that is, the user says the emergency voice key) or by button press (that is, the user presses the band emergency button), S61 is executed, including the following: the safety monitoring APP sends alarm information indicating that the user is in danger to the emergency contact in a form of a short message; the safety monitoring APP sends the alarm information indicating that the user is in danger to the public security alarm platform; trigger the positioning service, that is, the safety monitoring APP obtains the user's location information in real time and sends the location information to the emergency contact and the public security alarm platform; trigger an audio and video collection service, that is, the safety monitoring APP obtains video and voice message in real time and sends the information to the emergency contact and the public security alarm platform; and trigger a mutual assistance function, that is, if a person wearing the band is in an emergency situation, the safety monitoring APP sends a pop-up message saying “A user nearby triggered an emergency. If you encounter it, please offer help” to remind that a person living alone nearby may be in danger.

The personal safety assurance system provided in the present disclosure further includes a registration and login module. The registration and login module is configured for a new user to register and a registered user to log in. The new user needs to provide facial information, a name, a gender, an age, contact information, ID card information, an emergency contact number, the user's heart rate, blood pressure, and pulse information in normal state, and a preset emergency voice key to register. In addition, the user's name, gender, age, and facial image are instantly transmitted to the public security alarm platform, so that the police can quickly confirm the identity of an alarm reporter.

The personal safety assurance system further includes a punch-in module. The punch-in module includes a short message reminder module and a calling module. The user sets the daily punch-in time on the safety monitoring APP. The system is automatically awakened ten minutes before the punch-in time, and sends a message to remind the user to punch in. The band vibrates if the punch-in time approaches. In this case, the user only needs to shake the wrist three times to punch in. After the user punches in, the safety monitoring system is in a standby state to save power. If the user does not punch in in time, the system first calls the user. If the user does not answer the call, the system calls the emergency contact and asks the emergency contact to confirm the user's safety. The punch-in module is configured to remind the user to punch in, thereby ensuring the user's daily safety.

The personal safety assurance system further includes a mutual assistance module. When an emergency occurs, the band automatically sends a pop-up message saying “A user nearby triggered an emergency. If you encounter it, please offer help” to a nearby mobile phone with the same safety monitoring APP to remind that a person nearby may be in danger. This enhances the contact with the outside.

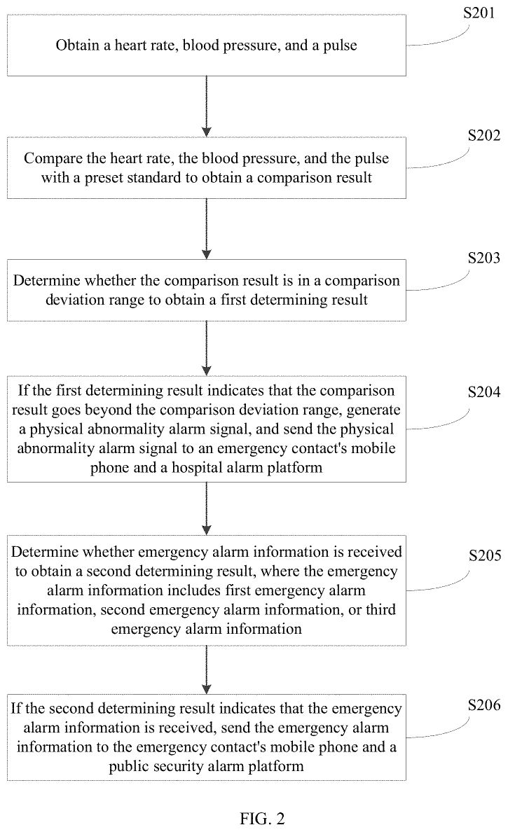

FIG. 2 is a flowchart of a safety monitoring method according to the present disclosure. The present disclosure further provides a safety monitoring method. As shown in FIG. 2, the monitoring method includes the following steps:

S201. obtain a heart rate, blood pressure, and a pulse.

S202. compare the heart rate, the blood pressure, and the pulse with a preset standard to obtain a comparison result.

S203. determine whether the comparison result is in a comparison deviation range to obtain a first determining result.

S204. if the first determining result indicates that the comparison result goes beyond the comparison deviation range, generate a physical abnormality alarm signal, and send the physical abnormality alarm signal to an emergency contact's mobile phone and a hospital alarm platform.

S205. determine whether emergency alarm information is received to obtain a second determining result, where the emergency alarm information includes first emergency alarm information, second emergency alarm information, or third emergency alarm information, specifically including the following:

if an alarm signal sent by a smart band 1 is received, the second determining result is that the first emergency alarm information is received;

if a voice message of a user is received, the voice message is matched with a preset emergency voice key, and if the matching succeeds, the second determining result is that the second emergency alarm information is received; or if a simulated call instruction of the user is received, dialog content is matched with the preset emergency voice key, and if the matching succeeds, the second determining result is that the third emergency alarm information is received. When receiving the simulated call instruction of the user, the safety monitoring system may provide dialog audio that imitates family members, friends, couples, etc. for the user to choose. During a simulated call, all content of a dialog provided by the system may be displayed on a screen in text format so that the user can simulate communication.

S206. if the second determining result indicates that the emergency alarm information is received, send the emergency alarm information to the emergency contact's mobile phone and a public security alarm platform.

After S204, the method may further include the following: obtain first positioning information, and send the first positioning information to the emergency contact's mobile phone and the hospital alarm platform.

After S206, the method may further include the following: obtain second positioning information, and send the second positioning information to the emergency contact's mobile phone and the public security alarm platform; and obtain video information and audio information, and send the video information and the audio information to the emergency contact's mobile phone and the public security alarm platform. Preferably, the audio information may be saved every ten seconds and uploaded to the cloud, and the audio information saved within ten minutes may be sent to the emergency contact.

The present disclosure provides the personal safety assurance system and the safety monitoring method, which can monitor the user in daily work and take countermeasures in emergencies. The system features a high degree of intelligence and can ensure that a distress signal or an alarm signal is transmitted effectively. In addition, the system has higher practical value and a wider application prospect especially for the increasing number of loners or people requiring improved security assurance.

The embodiments of the present disclosure are described above with reference to the accompanying drawings, but the present disclosure is not limited to the above specific embodiments. The above specific embodiments are merely illustrative and not restrictive. A person of ordinary skill in the art may make several modifications to the present disclosure without departing from the purpose of the present disclosure and the protection scope of the claims, and these modifications shall all fall within the protection of the present disclosure

What is claimed is:

1. A personal safety assurance system, comprising: a smart band and a safety monitoring application (APP), wherein
the smart band comprises a detection module, a main chip, a band emergency button, and a wireless band connection module;
an output end of the detection module is connected to an input end of the main chip, and the detection module is configured to detect a heart rate, blood pressure, and a pulse of a human body, and transmit the heart rate, the blood pressure, and the pulse to the main chip; an output end of the main chip is connected to the safety monitoring APP through the wireless band connection module; the main chip is configured to transmit the heart rate, the blood pressure, and the pulse to the safety monitoring APP through the wireless band connection module; the safety monitoring APP is configured to display the heart rate, the blood pressure, and the pulse, and convert the heart rate, the blood pressure, and the pulse into a physical abnormality alarm signal;
a signal output end of the band emergency button is connected to the input end of the main chip, and the band emergency button is configured to send an emergency alarm signal to the main chip; the main chip is further configured to transmit the alarm signal to the safety monitoring APP through the wireless band connection module; and
the safety monitoring APP is connected to a public security alarm platform and a hospital alarm platform through a network; and the safety monitoring APP is further configured to transmit the physical abnormality alarm signal to the hospital alarm platform and an emergency contact's mobile phone, generate emergency alarm information based on the emergency alarm signal, and transmit the emergency alarm information to the public security alarm platform and the emergency contact's mobile phone.

2. A safety monitoring method, comprising:
obtaining a heart rate, blood pressure, and a pulse;
comparing the heart rate, the blood pressure, and the pulse with a preset standard to obtain a comparison result;
determining whether the comparison result is in a comparison deviation range to obtain a first determining result;
if the first determining result indicates that the comparison result goes beyond the comparison deviation range, generating a physical abnormality alarm signal, and sending the physical abnormality alarm signal to an emergency contact's mobile phone and a hospital alarm platform;
determining whether emergency alarm information is received to obtain a second determining result, wherein the emergency alarm information comprises first emergency alarm information, second emergency alarm information, or third emergency alarm information; and
if the second determining result indicates that the emergency alarm information is received, sending the emergency alarm information to the emergency contact's mobile phone and a public security alarm platform.

3. The safety monitoring method according to claim 2, wherein the determining whether emergency alarm information is received to obtain a second determining result specifically comprises the following:
if an alarm signal sent by a smart band is received, the second determining result is that the first emergency alarm information is received;
if a voice message of a user is received, the voice message is matched with a preset emergency voice key, and if the matching succeeds, the second determining result is that the second emergency alarm information is received; or
if a simulated call instruction of the user is received, dialog content is matched with the preset emergency voice key, and if the matching succeeds, the second determining result is that the third emergency alarm information is received.

4. The safety monitoring method according to claim 2, after the sending the physical abnormality alarm signal to an emergency contact's mobile phone and a hospital alarm platform, further comprising:
obtaining first positioning information, and sending the first positioning information to the emergency contact's mobile phone and the hospital alarm platform.

5. The safety monitoring method according to claim 2, after the sending the emergency alarm information to the emergency contact's mobile phone and a public security alarm platform, further comprising:
obtaining second positioning information, and sending the second positioning information to the emergency contact's mobile phone and the public security alarm platform; and
obtaining video information and audio information, and sending the video information and the audio information to the emergency contact's mobile phone and the public security alarm platform.
